(19) United States
(12) Patent Application Publication
(10) Pub. No.: US 2014/0277178 A1
(43) Pub. Date: Sep. 18, 2014

(54) Posterior Ankle Fusion Plate

(71) Applicant: WRIGHT MEDICAL TECHNOLOGY, INC., Arlington, TN (US)

(72) Inventors: Timothy M. O'Kane, Munford, TN (US); Vinay D. Patel, Memphis, TN (US); Mary J. McCombs-Stearnes, Lakeland, TN (US); David Harness, Eads, TN (US)

(73) Assignee: Wright Medical Technology, Inc., Arlington, TN (US)

(21) Appl. No.: 13/801,018

(22) Filed: Mar. 13, 2013

(51) Int. Cl. A61B 17/80

(52) U.S. Cl. CPC A61B 17/8061
USPC 606/286

(57) ABSTRACT

An ankle arthrodesis fusion plate is provided that affords compression across the tibio-talar joint to promote bone fusion and joint stability.

### FIELD OF THE INVENTION

The invention relates to a device for fusing damaged, deteriorating, or fractured tibia, talus and calcaneus bones in the ankle region.

### BACKGROUND OF THE INVENTION

Arthrodesis refers to surgical fixation of a joint, ultimately resulting in bone fusion. Basically, the procedure is artificially induced ankylosis performed to relieve pain or provide support in a diseased or injured joint. Tibiotalocalcaneal or tibiocalcaneal arthrodesis (“TC”) is a salvage procedure for the treatment of joint disease or pain and dysfunction due to arthritic ankle and subtalar joints, e.g., Charcot disease. In performing ankle and subtalar arthrodesis, the surgeon typically wishes to achieve anatomic alignment, pain relief, and a stable, plantigrade foot. Attaining secure fixation while preserving the surrounding soft tissue is essential for a successful outcome.

A bone plate, of the type often used in TC, is a plate that is fastenable to the surface of a bone typically at both sides of a joint line to support and/or stabilize the joint. Bone plates have often been attached to the bone with bone screws that extend from the plate into the bone. In some examples, the head of the bone screw is locked to the plate (e.g., by threaded engagement between the screw head and the bone plate) and in other plates the head of the screw is free to angulate with respect to the plate, such that the screw may be placed in the bone at a surgeon-selected angle. In yet other examples, the screw head may cooperate with the bone plate to provide compression or distraction of the joint (i.e., to push the bone fragments towards or away from one another). Bone screws that angulate relative to the plate can often achieve less than adequate engagement with the bone which may severely limit secure fixation and lead to damage of surrounding soft tissue.

### SUMMARY OF THE INVENTION

An ankle arthrodesis system is provided that affords compression across the ankle and subtalar joint and associated bone structures of the lower leg, ankle, and foot to promote improved bone fusion and joint stability. In one embodiment, a fusion plate suitable for receiving bone anchors is provided that includes a shaft having a proximal portion, a distal portion, and a longitudinal axis. A plurality of through-bores are defined within the proximal portion, and a transverse contour of the proximal portion is defined by a first radius and a longitudinal contour of the proximal portion is defined by a first angle. The first angle and the first radius are selected so as to provide effective positioning of the distal portion for tibiotalocalcaneal fixation. The distal portion is arranged so as to be flared away from the a longitudinal axis. The flare is defined by a second angle. The distal end also includes a plurality of eyelets each being suitable for receiving a bone anchor and each offset from the longitudinal axis. The transverse contour of the distal portion is defined by a second radius where the second angle and the second radius are selected so as to provide delimited angulated access of the bone anchor to each eyelet such that each bone anchor will achieve effective bone purchase and thereby secure tibiotalocalcaneal fixation.

### BRIEF DESCRIPTION OF THE DRAWINGS

These and other features and advantages of the present invention will be more fully disclosed in, or rendered obvious by, the following detailed description of the preferred embodiments of the invention, which are to be considered together with the accompanying drawings wherein like numbers refer to like parts and further wherein:

FIG. 1 is a lateral view of a left foot, ankle, and lower leg of a human skeleton;

FIG. 2 is a perspective view of a fusion plate formed in accordance with the present invention;

FIG. 3 is a front plan view of the fusion plate shown in FIG. 2;

FIG. 4 is a broken-away, end-on perspective view, partially in cross-section, of a distal end of the fusion plate, as taken along line 4-4 in FIG. 3;

FIG. 5 is a broken-away, perspective view of an eyelet portion of the distal portion shown in FIG. 4, illustrating one aspect of the delimited angular relationship between a bone screw and a through-bore in the eyelet;

FIG. 6 is a perspective end-on view of a distal portion of a fusion plate formed in accordance with the present invention and assembled to a portion of the calcaneous showing bone anchors in a converging orientation illustrating an aspect of the delimited angular relationship between bone anchors, eyelets, and the calcaneous;

FIG. 7 is a perspective end-on view, similar to FIG. 6, showing bone anchors in a diverging configuration illustrating another aspect of the delimited angular relationship between bone anchors, eyelets, and the calcaneous;

FIG. 8 is a perspective end-on view similar to FIGS. 6 and 7, but showing the bone screws in a parallel configuration illustrating a further aspect of the delimited angular relationship between bone anchors, eyelets, and the calcaneous;

FIG. 9 is a perspective top end-on view of a fusion plate formed in accordance with the present invention and assembled to a portion of the talus showing bone anchors in a converging orientation illustrating an aspect of the delimited angular relationship between bone anchors, eyelets, and the talus;

FIG. 10 is a perspective top end-on view, similar to FIG. 9, showing bone screws in a diverging configuration illustrating another aspect of the delimited angular relationship between bone anchors, eyelets, and the talus;

FIG. 11 is a perspective top end-on view, similar to FIGS. 9 and 10, but showing the bone screws in a parallel configuration illustrating a further aspect of the delimited angular relationship between bone anchors, eyelets, and the talus;

FIG. 12 is a side perspective view of a fusion plate illustrating angular relationships between the proximal and distal portions of the fusion plate;

FIG. 13 is a lateral view of a partial foot, ankle, and lower leg of a human skeleton as shown in FIG. 1, having a fusion plate secured to the tibia, talus, and calcaneous in accordance with the invention;

FIG. 14 is a rear view of the fusion plate and antero-lateral left foot shown in FIG. 1, having a fusion plate secured to the tibia, talus, and calcaneous in accordance with the invention;

FIG. 15 is a side elevational view illustrating engagement a fusion plate secured to the tibia, talus, and calcaneous in accordance with the invention, where the bone is illustrated so as to be transparent thereby revealing portions of a screw located within it;

FIG. 16 is a rear perspective view of the foot, ankle and leg skeleton fused with a fusion plate in accordance with the present invention; and

FIG. 17 is another rear perspective view of the foot, ankle and leg skeleton fused with a fusion plate in accordance with the present invention, where the bone is illustrated so as to be transparent thereby revealing portions of a screw located within it.

### DETAILED DESCRIPTION OF THE PREFERRED EMBODIMENT

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. When only a single machine is illustrated, the term “machine” shall also be taken to include any collection of machines that individually or jointly execute a set (or multiple sets) of instructions to perform any one or more of the methodologies discussed herein. In the claims, means-plus-function clauses, if used, are intended to cover the structures described, suggested, or rendered obvious by the written description or drawings for performing the recited function, including not only structural equivalents but also equivalent structures.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. The term “and/or” is used in the same manner, meaning “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

FIG. 1 provides a lateral view of the skeleton of a foot, ankle, and distal leg portion that includes tibia A and fibula B. The bones of the ankle and foot include: talus C, calcaneus D, navicular E, cuboid F, cuneiforms G, metatarsals H, and phalanges I. The sinus tarsi J is a canal-like space formed between the inferior surface of the talus at the sulcus tali and the superior surface of the calcaneus at the calcaneal sulcus.

Referring to FIGS. 2-4, fusion plate 2 has a longitudinal axis 4 and a shaft 6. Shaft 6 defines a plurality of through-bores 8 along a proximal portion 9 and includes off-set eyelets 10 along a distal portion 11. Fusion plate 2 may be constructed from biocompatible materials such as, for example, titanium, alloys of titanium, stainless steel, resorbable materials such as polymers and allograft, although one of ordinary skill in the art will know and appreciate that any biocompatible material may be used. Plurality of through-bores 8 each define a central axis 12, and are configured to receive a bone anchor, often in the form of a bone screw 13 (FIG. 5). Typically, the interior surfaces of shaft 6 that define a through-bore 8 include a threaded portion 15. Of course, other types of bone anchors known to one of ordinary skill in the art, such as blades, nails, pins, etc., may be used to achieve adequate results. Often, bone screw 13 may be constructed from, for example, titanium, alloys of titanium, stainless steel, resorbable materials such as polymers, allograft or other biocompatible materials known in the art. Bone screw 13 is preferably compatible with fusion plate 2 in terms of composition and strength. Bone screw 13 may be cannulated having a through-bore or channel (not shown) extending from the upper surface of its head to the tip, for introducing instruments, for example, a guide wire into the joint line. The engagement of fusion plate 2 and bone screws 13 effectively anchors fusion plate 2 to the posterior portions of calcaneous, talus, and tibia so as to fuse them together (FIGS. 13-17).

Shaft 6 of fusion plate 2 is defined by at least two radii. A transverse radius R17 is defined along proximal portion 9 of shaft 6 (FIG. 4). In preferred embodiments, transverse radius R17 is in the range from about 0.090 inches to about 1.10 inches, with about 1.0 inch being preferred for most applications of the invention. Transverse radius R17 defines the contour of the portion of shaft 6 that engages tibia A and talus C. There is also a second transverse radius R19 defined along distal portion 11 of shaft 6 that provides clearance for close engagement of distal portion 11 with talus C and calcaneous D (FIGS. 2, 6-8, and 9-11). Transverse radius R19 is in the range from about 0.055 inches to about 0.65 inches, with about 0.60 inch being preferred for most applications of the invention. Transverse radius R19 defines the contour of the portion of shaft 6 that engages calcaneous D. These radii are optimized to allow each bone screw 13 to purchase bone effectively at extreme angles, as described herein below in further detail (FIGS. 6-11). In addition, distal portion 11 is flared away from longitudinal axis 4 by an angle β, in the range from about 25° to about 35°, with about 30° being preferred for most applications of the invention. An angle θ measured from the region of shaft 6 that begins its β degree talocalcaneal flare and the end of proximal portion 9 of shaft 6 defines the longitudinal contour of proximal portion 9. Angle θ is often in the range from about 93° to about 97°, with about 95° being preferred for most applications of the invention (FIG. 12). The selection of radius R19 and angle θ provides effective positioning of distal portion 11 for tibiotalocalcaneal fixation. In preferred embodiments, the total included angle (β+θ) over the length of fusion plate 2 is in the range from about 122° to about 127°, with about 125° being preferred for most applications of the invention. These angular relationships likewise ensure that each bone screw 13 advantageously purchases bone at extreme angles, as described herein below in further detail.

Referring to FIGS. 3-11, off-set eyelets 10 project outwardly from the edges of distal 11 portion of shaft 6 so as to straddle longitudinal axis 4. The bottom surfaces of off-set eyelets 10 are often located adjacent to posterior aspects of talus C and calcaneus D (FIGS. 6-17). More particularly, a first pair of talar-eyelets 10a and 10b each define a threaded through-bore 18 and a second pair of calc-eyelets 10c and 10d each define a threaded through-bore 19. Threaded through-bore 18 of each talar-eyelet 10a and 10b and calc-eyelet 10c and 10d defines a central axis 22 (FIGS. 4 and 5). Talar-eyelets 10a and 10b each accept a bone screw 13 within a purchase-cone 25 that is advantageously defined as a solid angle of revolution about central axis 22 at an angle α, in the range from about 13° to about 17°, with about 15° from central axis 22 being preferred for most applications of the invention (FIGS. 4 and 5). Often, purchase-cone 25 defines a total included angle about central axis 22 of approximately 30° so as to delimit angulated access of a bone anchor to each eyelet such that each bone anchor achieves effective bone purchase, thereby improving tibiotalocalcaneal fixation by fusion plate 2.

Talar-eyelets 10a and 10b and calc-eyelets 10c and 10d are each configured for engaging the head of bone screw 13. More preferably, talar-eyelets 10a and 10b and calc-eyelets 10c and 10d define threaded through-bores 18 and 19 that may be configured for fixing and locking with bone screw 13 and more preferably for fixing bone screw 13 in a fixed, predetermined orientation with respect to the lower surface of fusion plate 2 or the exterior surface of talus C and calcaneus D into which it is driven. For example, such fixation may be by threaded engagement, interference or press fitting, or any other form of joining talar-eyelets 10a and 10b and calc-eyelets 10c and 10d with the screw heads known to one of ordinary skill in the art. Bone screw 13 is fixed to talar-eyelets 10a and 10b and calc-eyelets 10c and 10d of fusion plate 2 such that its shaft or shank would extend within purchase-cone 25.

Placement of bone screws 13 within purchase-cone 25, will ensure that bone screws 13 always purchase the bones of talus C and calcaneus D, i.e., always acquire a leveraged and secure engagement between the threads on the surface of bone screw 13 and the interior of the bone as the screw is rotated inwardly toward the bone. If in an extreme placement, bone screws 13 in calc-eyelets 10c and 10d are oriented at shallow, converging angles, e.g., an angle μ measured between the bone screws in the range from about 20° to about 24°, with about 22° being typical (FIG. 6) the intersection point of bones screws 13 is deeper in the bone, with sufficient purchase to ensure secure engagement. However, if bone screws 13 are diverging, e.g., an angle μ measured between the bone screws in the range from about 80° to about 84°, with about 82° being typical (FIG. 7) bones screws 13 are shallower and closer to the surface of calcaneous D, but with sufficient purchase to ensure secure engagement. If bone screws 13 in calc-eyelets 10c and 10d are placed in-line with central axis 22 of through-bore 18, bone screws 13 in calc-eyelets 10c and 10d are essentially parallel to one another and again with sufficient purchase to ensure secure engagement (FIG. 8).

Referring to FIGS. 9-11, talar-eyelets 10a and 10b also define a purchase-cone that will ensure that bone screws 13 always purchase talus C, i.e., always acquire a leveraged and secure engagement between the threads on the surface of bone screw 13 and the interior of the bone as the screw is rotated inwardly toward the bone. If in an extreme placement, bone screws 13 in talar-eyelets 10a and 10b are oriented at shallow, converging angles, e.g., an angle μ measured between the bone screws in the range from about 20° to about 24°, with about 22° being typical (FIG. 9) the intersection point of bones screws 13 is deeper in the bone, with sufficient purchase to ensure secure engagement. However, if bone screws 13 are diverging, e.g., an angle μ measured between the bone screws in the range from about 80° to about 84°, with about 82° being typical (FIG. 10) bones screws 13 are shallower and closer to the surface of talus C, but with sufficient purchase to ensure secure engagement. If bone screws 13 in talar-eyelets 10a and 10b are placed in-line with central axis 22 of through-bore 18, bone screws 13 in talar-eyelets 10a and 10b are essentially parallel to one another and again with sufficient purchase to ensure secure engagement (FIG. 11).

It is to be understood that the present invention is by no means limited only to the particular constructions herein disclosed and shown in the drawings, but also comprises any modifications or equivalents within the scope of the claims.

What is claimed is:

1. A fusion plate suitable for receiving bone anchors for use in tibiotalocalcaneal or tibiocalcaneal arthrodesis comprising:
a shaft having a proximal portion, a distal portion, a longitudinal axis, and a plurality of through-bores defined within said proximal portion wherein a transverse contour of said proximal portion is defined by a first radius and a longitudinal contour of said proximal portion is defined by a first angle, wherein said first angle and said first radius are selected so as to provide effective positioning of said proximal portion for at least one of tibiotalocalcaneal and tibiocalcaneal fixation;
said distal portion arranged so as to be flared away from said longitudinal axis said flare being defined by a second angle, and including a plurality of eyelets each being suitable for receiving a bone anchor and that are each offset from said longitudinal axis wherein a transverse contour of said distal portion is defined by a second radius wherein said second angle and said second radius are selected so as to provide delimited angulated access of said bone anchor to each said eyelet such that each said bone anchor may achieve effective bone purchase and thereby secure at least one of tibiotalocalcaneal and tibiocalcaneal fixation.

2. A fusion plate according to claim 1 wherein said first radius is in the range from about 0.090 inches to about 1.10 inches.

3. A fusion plate according to claim 1 wherein said first radius is about 1.0 inches.

4. A fusion plate according to claim 1 wherein said second radius is in the range from about 0.055 inches to about 0.65 inches.

5. A fusion plate according to claim 1 wherein said second radius is about 0.60 inches.

6. A fusion plate according to claim 1 wherein said distal portion is flared away from said longitudinal axis by an angle in the range from about 27° to about 33°.

7. A fusion plate according to claim 1 wherein said distal portion is flared away from said longitudinal axis by an angle of about 30°.

8. A fusion plate according to claim 1 wherein said first angle is in the range from about 93° to about 97°.

9. A fusion plate according to claim 1 wherein said first angle is about 95°.

10. A fusion plate according to claim 1 wherein said off-set eyelets project outwardly from edges of said distal portion of said shaft so as to straddle said longitudinal axis.

11. A fusion plate according to claim 10 wherein a first pair of talar-eyelets each define a threaded through-bore and a second pair of calc-eyelets each define a threaded through-bore said through-bores defining a central axis such that a solid angle of revolution about said central axis at an angle in the range from about 13° to about 17° which defines a purchase-cone.

12. A fusion plate according to claim 10 wherein a first pair of talar-eyelets each define a threaded through-bore and a second pair of calc-eyelets each define a threaded through-bore said through-bores defining a central axis such that a solid angle of revolution about said central axis at an angle of about 14° to 15° which defines a purchase-cone.

13. A fusion plate according to claim 12 wherein placement of said bone anchor within said purchase-cone delimits angulated access of said bone anchor to each said off-set eyelet such that each bone anchor achieves effective engagement with an underlying bone.

14. A fusion plate suitable for receiving bone anchors for use in at least one of tibiotalocalcaneal and tibiocalcaneal arthrodesis comprising:
a shaft having a proximal portion, a distal portion, a longitudinal axis, and a plurality of threaded through-bores defined within said proximal portion wherein a transverse contour of said proximal portion is defined by a first radius and a longitudinal contour of said proximal portion is defined by a first angle, wherein said first angle and said first radius are selected so as to provide effective positioning of said proximal portion for at least one of tibiotalocalcaneal and tibiocalcaneal fixation;
said distal portion arranged so as to be flared away from said a longitudinal axis said flare being defined by a second angle, and including a plurality of eyelets each being suitable for receiving a bone anchor and that are each offset from said longitudinal axis wherein a transverse contour of said distal portion is defined by a second radius that is less than said first radius wherein said second angle and said second radius are selected so as to provide delimited angulated access of said bone anchor to each said eyelet such that each said bone anchor may achieve effective bone purchase and thereby secure tibiotalocalcaneal fixation.

15. A fusion plate according to claim 14 wherein said off-set eyelets project outwardly from edges of said distal portion of said shaft so as to straddle said longitudinal axis.

16. A fusion plate according to claim 15 wherein a first pair of talar-eyelets each define a threaded through-bore and a second pair of calc-eyelets each define a threaded through-bore said through-bores defining a central axis such that a solid angle of revolution about said central axis at an angle in the range from about 13° to about 17° which defines a purchase-cone.

17. A fusion plate according to claim 14 wherein a first pair of talar-eyelets each define a threaded through-bore and a second pair of calc-eyelets each define a threaded through-bore said through-bores defining a central axis such that a solid angle of revolution about said central axis at an angle of about 14° to 15° which defines a purchase-cone.

18. A fusion plate according to claim 16 wherein placement of said bone anchor within said purchase-cone delimits angulated access of said bone anchor to each said off-set eyelet such that each bone anchor achieves effective engagement with an underlying bone.
